(19) United States
(12) Patent Application Publication
(10) Pub. No.: US 2007/0078365 A1
(43) Pub. Date: Apr. 5, 2007

(54) Adhesive bandage product and manufacturing process for an adhesive bandage product

(76) Inventor: Carlos Macedo, Sao Jose dos Campos, SP (BR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/398,310

(22) Filed: Apr. 5, 2006

(30) Foreign Application Priority Data
Oct. 4, 2005 (BR) BR PI0404697-8

(51) Int. Cl. A61F 15/00

(52) U.S. Cl. 602/52

(57) ABSTRACT

This invention covers an adhesive bandage product that consists of at least an initial layer of structural film 1, at least one adhesive coating 2, associated with the initial layer of structural film 1 and a pad 3, and a cavity 6 shaped by the initial layer of plastic film 1 for the placement of the pad.

The invention also covers a process for the manufacturing of this adhesive bandage product, consisting of Step (i) for plastically shaping a cavity 6 at least partially under pressure in the initial layer of the structural film 1, coated, for the placement of a pad 3 therein.

This invention relates to an adhesive bandage product especially designed for use on the skin, in addition to a process for the manufacturing thereof.

### DESCRIPTION OF THE STATE OF THE ART

Conventionally, adhesive bandages have been used on the skin in order to protect wounds from dirt and ensure the efficacy of topical medications, consisting of an adhesive film associated with a pad of absorbent material, which is the element to be placed over the cut, ensuring that the wound is protected and reducing discomfort as much as possible through its use.

In order to accelerate the healing of the wound and allow the skin to breathe, the adhesive film is preferably pierced by many orifices.

Although this basic configuration has been known for many years, it is extremely efficient, which is why upgrading this type of adhesive bandage has taken place through the use of new materials that are more flexible, comfortable, low-cost etc., as well as through alterations in the coloring of the adhesive bandage (skin color, transparent, colored, decorated with cartoon characters, etc.).

Nevertheless, regardless of the specific elements of their color, material and shape, the characteristics of all adhesive bandages have been essentially the same.

An improvement in this type of product may be found in U.S. Pat. No. 5,928,972, which covers an adhesive bandage and a method for the manufacturing thereof.

The adhesive bandage covered by this document consists of a plastic film that constitutes the body of the adhesive bandage, an absorbent pad for placing over the wound, and as the major innovation, a layer of woven or non-woven fabric consisting at least partially of polymer material. The ends of the fabric or non-fabric layer are fused to the plastic film, and the adhesive layer is positioned on the fabric or non-fabric layer. The main advantages offered by this adhesive bandage are the facts that it maximizes adherence to the skin while ensuring, in counterpart, ample cutaneous respirability.

Additionally, all adhesive bandages known to date present an undesirable characteristic, which is that they do not ensure full isolation of the wound from outside elements, particularly when located close to the joints of the body (near the knee, elbow, fingers etc.). This inconvenience is due to the fact that in the regions around the absorbent pad, the area of film coated with adhesive is narrow, and the presence of the pad (which is compressed so that the film around it enters into contact with the skin) generates, a traction force that tends to loosen the film. When the adhesive bandage is close to joints, this disadvantage is far more serious, as the movement of the limbs of the user loosens the above-mentioned portions of the film alongside the pad almost immediately.

The consequences of this loosening may be, among others:

possibility of contamination of the wound by dirt;

possibility that dirt may stick to the plastic film in the loosened regions, giving rise to a “dirty” appearance that results in the adhesive bandage being replaced when not necessary; and

impression of poor quality of the adhesive bandage among users/consumers;

should it not loosen, the pad places pressure on the injury, due to the pressure imposed-by the adhesive element (glue).

Consequently, so far no disposable adhesive bandage had been developed that does not present the inconvenient aspects listed above.

### OBJECTIVES OF THE INVENTION

The primary objective of the invention is to supply an adhesive bandage product that continues to adhere easily to the skin of the user, without the portions alongside the absorbent pad coming loose due to areas of tension on its application surface, such as the injured or wounded skin, and consequently preventing the entry of dirt, remaining firm during its use.

A second objective of the invention is to supply a process for the manufacturing of the adhesive bandage product covered above.

### BRIEF DESCRIPTION OF THE INVENTION

The primary objective of the invention is achieved through an adhesive bandage product consisting of at least one initial layer of structural film, at least one adhesive coating associated with the initial layer of structural film, at least one pad and at least one cavity contoured in the initial layer of plastic film in which the pad is placed.

The second objective of the invention is achieved through a process for the manufacturing of an adhesive bandage product, particularly a product as defined above, consisting of the following steps:

(i) applying an adhesive coating to an initial layer of structural film;

(ii) positioning at least one pad on the initial layer of the structural film; and

(ii) shaping at least partially and plastically under pressure, forming at least one cavity in the initial layer of the structural film coated in step (i).

This invention offers as advantages the fact that it results in an adhesive bandage that ensures that the wound is kept completely isolated from outside elements, even when used close to the joints of the body (near the knee, elbow, on the fingers etc.), due to the fact that the pad does not generate forces that result in the adhesive bandage becoming unstuck.

For the same reason, the pad does not press on the wound, lessening the pain felt by the user.

Moreover, this characteristic increases the length of time of use of the adhesive bandage, as the wound and the pad are not contaminated by dirt.

### BRIEF DESCRIPTION OF THE DRAWINGS

This invention will be described in greater detail below, based on an example produced and represented in the drawings. The Figures show:

FIG. 1—an initial view in perspective of the adhesive bandage product covered by this invention;

FIG. 2—corresponds to two lengthways cross-sections of the adhesive bandage product illustrated in FIG. 1, following the A-A and B-B lines;

FIG. 3—this is a partial perspective of the adhesive bandage product covered by this invention, showing the covering layer that provides protection for the adhesive coating;

FIG. 4—is a detailed cross-section view of the adhesive bandage product covered by this invention; and

FIG. 5—is a detailed cross-section view of the adhesive bandage product of the technical state of the art.

### DETAILED DESCRIPTION OF THE FIGURES

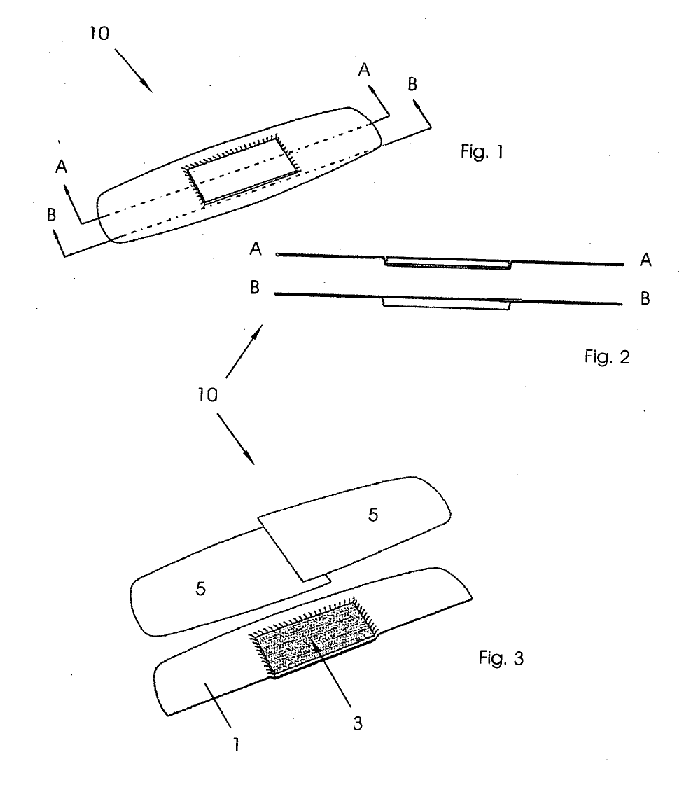

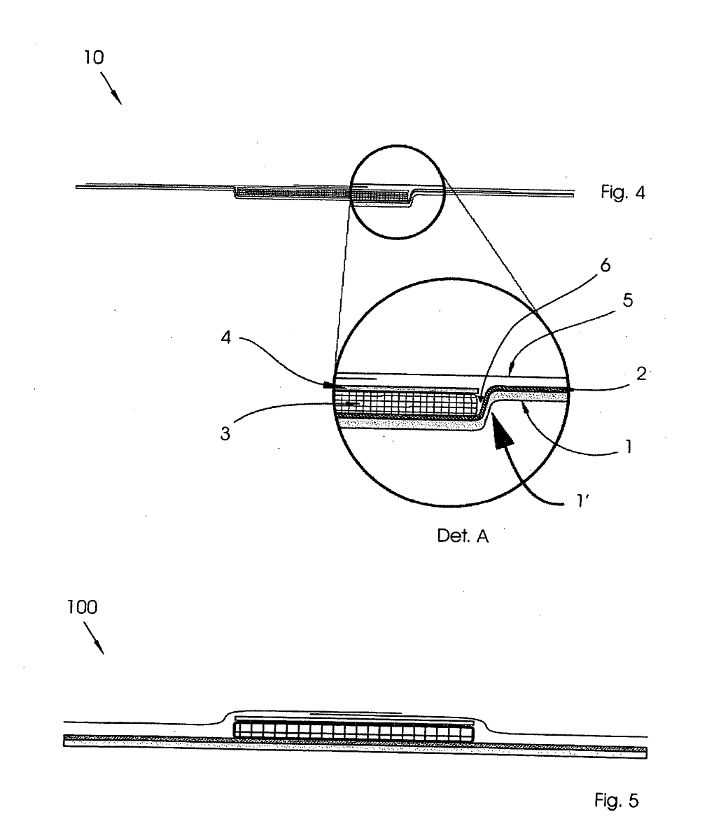

As may be seen in FIGS. 1-4, adhesive bandage product 10 covered by this invention consists essentially of at least an initial layer of structural film 1, defining an initial surface that will appear on the skin of the user of the adhesive bandage 10, and a second surface, on the other side, with which at least one adhesive 2 coating is associated, and at least one pad 3, positioned in a cavity 6 contoured on the film 1.

Film 1 may take various shapes, such as the preferential format, substantially rectangular with secondary rounded corners, the circular or square formats, or any other shape that may be necessary and/or desirable.

Preferably, film 1 consists of polyethylene (PE) although this may consist of any other desired polymer, such as polypropylene (PP), vinyl polychloride, foamed polyurethane, non-foamed polyester, combinations thereof and others.

Still in a preferential manner, film 1 is pierced by many orifices (not illustrated) to allow cutaneous respirability when the adhesive bandage 10 is being worn. These openings may be positioned in any pattern whatsoever over all or part of the area of the film 1, and may also simply not be provided.

Film 1 serves as a support layer for handling by the user, and as a support for the additional layers of the adhesive bandage product. It should be flexible enough to allow the adhesive bandage product to adjust comfortably to the different parts of the body.

The initial surface of the structural film 1 is associated with at least one adhesive coating 2, preferably in the form of an adhesive layer that covers its entire area. Obviously, the adhesive 2 coating used may be of any type whatsoever, in addition to its specific constitution. As an alternative, for instance, an adhesive coating 2 may be planned to consist of many lines of adhesive, or any other possible situation.

Preferably, the adhesive coating corresponds to a layer 2 of adhesive, of a type known to technical experts in this matter as “hot melt”, and may have an acrylic composition, or be silicon-based, water-based, polyurethane, SEEPS, or any other necessary or desirable composition. The portion of the adhesive coating 2 located outside cavity 6 is able to adhere to an application surface for the adhesive bandage product 10.

Adhesive bandage 10 also consists of at least one pad 3 or any other equivalent elements whose function is to touch the wound, absorbing exudates expelled by the body, such as blood, pus and other liquids. Additionally, pad 3 allows prolonged contact by topical medications (such as creams and ointments) with the wound covered by the surface of the adhesive bandage 10 to which they are applied.

The pad 3 consists preferably of a rayon-based non-woven fabric, (which is a cellulose material with good absorption properties) and polyester, but it is clear that its composition may be any other, provided that this is functional. The pad 3 may be made through a variety of manufacturing processes, such as spunlace, spunbonding, needle punch, etc.

Pad 3 may be any shape, provided that it is functional. In the preferential example of this invention, pad 3 is largely rectangular.

Pad 3 includes, also on a preferential basis, a second layer of an additional second non-adherent and perforated plastic film 4, designed to prevent it from sticking to the wound, which might be unpleasant or painful when the adhesive bandage must be removed. Film 4 consists preferably of polyethylene (PE), polypropylene (PP), vinyl polyichoride (PVC), foamed polyurethane, non-foamed polyester or combinations thereof. The plastic film 4 may be coated with additives such as bactericide and germicide elements, such as silver colloids. However film 4 is merely optional.

Still on a preferential basis, the adhesive bandage product 10 includes an additional covering layer 5 consisting of two segments of film placed partially over the adhesive coating and the pad 3, in order to insulate the dressing from outside elements and keep the adhesive effectively sticky. The covering layer 5 should be removed when the adhesive bandage 1 is to be used, which is why it should be easy to handle (removable).

The covering layer 5 consists preferably of a silicone polyethylene film, although polypropylene or polystyrene films may be used, or combinations thereof, silicon-coated papers or any other functional elements.

The main innovation of the adhesive bandage 10 covered by this invention, which endows it with countless advantages as will be described below, lies in the fact that, in contrast to the adhesive bandages currently known, film 1 has a cavity 6 for positioning the pad. Consequently, cavity 6 is contoured by the initial layer of plastic film 1 for the placement of the pad 3.

The cavity 6 allows the pad 3 to be positioned in a manner whereby its outer side facing the body of the user is leveled with that of the rest of the film 1 area not including the cavity. The process for producing the cavity will be explained a posteriori, but this effectively corresponds to a portion of film 1 that is shaped by stretching, so that it takes on the permanent shape of a cavity, without tending to return to its initial shape, level with the rest of film 1. Due to this stretching process, the cavity walls—shown in the Figures as 1′—are stretched compared to the rest of film 1, with a reduction in thickness arising from the plastic shaping process of this region.

The fact that the entire contact surface with pad 3 other than cavity 6 is in contact with the rest of the film 1 means that when the adhesive bandage is placed on the body of the user to cover a wound, there is no compression of the pad. As a result, the pad 3 does not impose pressure that tends to loosen the film 1 from the skin of the user in the surrounding regions. As a result, when the adhesive bandage 10 is applied, there is no build-up of tensions in the walls 1′ of the cavity 6. The result is that the regions of the film alongside the pad 3 remain glued to the skin, stopping dirt from entering the wound, and also preventing adhesive bandage 10 from becoming dirty.

These properties are maintained, even when adhesive bandage 10 is applied to parts of the user's body where there are joints, such as knees, elbows, fingers, etc., as pad 3 is always free from compression forces, which is why there are no forces that will tend to loosen the film from the skin. Similarly, the pad 3 does not press on the wound, reducing the pain felt by the user.

In the adhesive bandage 100 of the technical state of the art shown in FIG. 5, the film is substantially flat, with no cavity, and the pad is simply placed on it. This means that when the adhesive bandage is used, the thickness of the pad will be compressed, so that the areas alongside it touch the skin and remain in this position, preventing dirt from entering the wound. However, the pad will tend to return to its initial position, forcing the areas of the film alongside it to become unglued. This situation becomes worse when the adhesive bandage is placed on or near the joints of the body, whose movements help speed up this loosening process, which is almost immediate.

Preferably, cavity 6 presents a shape that is substantially equivalent to that presented by pad 3 in both area and height, but it is clear that an adhesive bandage may be designed whose cavity 6 has an area and/or height greater than that corresponding to the area and thickness of the pad 3. Consequently, cavity 6 may have any shape, provided that it is functional, and that pad 3 does not impose forces that loosen the areas of film 1 alongside it.

An adhesive bandage 10 may also be designed containing more than one pad 3 in each cavity 6, as well as an adhesive bandage 10 containing various cavities 6, each with one or more pads 3, or even some other configuration, provided that it is functional, all of which are covered in the scope of the protection offered by the claims in question.

The manufacturing process for the adhesive bandage 10 product described above is also a new and inventive invention, consisting of the following steps:

Step (i): application of an adhesive 2 coating to an initial layer of structural film 1;

Step (ii): placing at least one pad 3 on the initial layer of structural film 1 coated in Step (i); and

Step (iii): plastic shaping, at least partially under pressure of at least one cavity 6 in the initial layer of the structural film 1 coated in Step (i).

The shaping Step (iii) is preferably undertaken by punching. A punch is used to punch out the pad 3 that plastically reshapes the film 1 in the region where it is placed, forming cavity 6. With this reshaping process, the plastic film 1 is stretched, corresponding to the walls 1′ of the cavity, which are given a new length, greater than initially. The reshaping is plastic, meaning that the walls 1′ do not return to their initial position after punching. The punching may be undertaken through either hot or cold processing.

Preferably, pad 3 is placed on film 1 (Step (ii)) before punching (Step (iii)). However, alternatively, a manufacturing process may be designed where the pad is positioned concomitantly with the punching, and when Steps (ii) and (ii) occur substantially concomitantly. The results obtained are identical.

Generically, Step (ii) then consists of placing at least one pad 3 on film 1 previously or substantially concomitantly to the punching process.

Evidently, a configurative variant of this process may be foreseen, in which the punching takes place prior to the positioning of the pad, in addition to which a manufacturing process is also possible in which the punching takes place prior to the application of the adhesive to film 1.

Moreover, a variant of the manufacturing process may be established through which cavity 6 is shaped not by punching but rather through in any other possible method, such as vacuum suction shaping.

The process covered by this invention also consists of a step during which a second layer of non-adherent, perforated layer of plastic film 4 is placed on pad 3, with this step undertaken prior to the cavity shaping (Step (iii)).

The process covered by this invention may also include a Step through which a cover layer 5 is applied to the initial structural layer 1 and pad 3, after Step (iii).

Having described a preferred specific embodiment, it should be understood that the scope of this invention covers other possible variations, being limited only by the content of the claims appended hereto, including the possible equivalence thereof.

1. Adhesive bandage product characterized by the fact that it consists of:
At least an initial layer of structural film (1);
At least one adhesive coating (2) associated with the initial layer of structural film (1); and
At lease one pad (3);
the product is characterized by the fact that it consists of one cavity 6 contoured by the initial layer of plastic film 1 for placement of the pad 3.

2. Adhesive bandage product as covered by claim 1, characterized by the fact that it additionally consists of a second layer of non-adherent, perforated plastic film 4, placed over the pad 3.

3. Adhesive bandage product according to claim 1 or 2, characterized by the fact that it additionally includes a covering layer 5.

4. Adhesive bandage product according to any of the claims 1-3, characterized by the fact that the part of the adhesive coating 2 located outside the cavity 6 is able to adhere to a surface for the application of the product.

5. Adhesive bandage product according to any of the claims 1-4, characterized by the fact that the initial layer of structural film 1 and the second layer of non-adherent, perforated plastic film 4 are made of polyethylene, polypropylene, vinyl polychloride, foamed polyurethane, non-foamed polyester or combinations thereof.

6. Adhesive bandage product according to claims 1-5, characterized by the fact that the pad 3 is made of rayon, polyester or combinations thereof.

7. Adhesive bandage product according to any of the claims 1-6, characterized by the fact that the covering layer 5 is made of polyethylene, polypropylene, polystyrene or combinations thereof.

8. Process for the manufacturing of an adhesive bandage product, particularly a product as defined in any of claims 1-7, characterized by the fact that it includes the following Steps:
(i) application of an adhesive coating 2 to an initial layer of structural film 1 (1);
(ii) placing at least one pad 3 on the initial layer of structural film 1; and
(iii) plastically shaping at least partially and under pressure at least one cavity 6 in the initial layer of structural film 1 coated in Step (i).

9. Method, according to claim 8, characterized by the fact that Step (iii) of the shaping is handled through a punching process.

10. Method, according claims 8-9, characterized by the fact that the punching is handled through either a hot or cold process.

11. Method, according to any of the claims 8-10, characterized by the fact that after Step (iii) of the shaping, the cavity 6 remains permanently shaped.

12. Method, according to any of the claims 8-11, characterized by the fact that it additionally includes a step of applying a second layer of plastic, non-adherent and perforated film 4, placed over the pad 3 prior to Step (iii).

13. Method, according to any of the claims 8-12, characterized by the fact that it additionally includes a step of applying a covering layer 5 to the initial structural layer 1 and over the pad 3, after Step (iii).

14. Method, according to claim 8, 9, 10 or 11, characterized by the fact that Steps (ii) and (iii) are substantially concomitant.
